(19) United States
(12) Patent Application Publication
(10) Pub. No.: US 2011/0034763 A1
(43) Pub. Date: Feb. 10, 2011

(54) Anthropometric Massage Device

(76) Inventors: Lauren Domnick, New Berlin, WI (US); Troy Domnick, New Berlin, WI (US)

Correspondence Address: HARSHAW RESEARCH, INC., 210 W. TECUMSEH STREET, OTTAWA, KS, 66067, US

(21) Appl. No.: 12/850,268

(22) Filed: Aug. 4, 2010

Related U.S. Application Data
(60) Provisional application No. 61/231,398, filed on Aug. 5, 2009.

(51) Int. Cl. A61F 5/00; A61H 1/00

(52) U.S. Cl. 600/38; 601/46

(57) ABSTRACT

An anthropometric massage device includes a length adjustable handle having first and second portions slidably movable relative to one another for telescopically varying the length of the handle. A shape adjustment member having a plurality of rotatable joints extending between opposed ends thereof for movement between straight and curved configurations. The rotatable joints are secured at selected positions in a friction fit engagement. The massage device includes a stimulation member having opposed ends and a cylindrical configuration defining an open interior space. A vibrating motor is positioned within the interior space of the stimulation member and is electrically connected to a power source, the stimulation member being vibrated when the motor is actuated.

### REFERENCE TO RELATED APPLICATIONS

The present invention claims the benefit of U.S. Provisional Patent Application Ser. No. 61/231,398, filed on Aug. 5, 2009 titled “Anthropometrically Designed Personal Device.”

### BACKGROUND OF THE INVENTION

This invention relates generally to massage devices and, more particularly, to a massage device configured to adjust easily to a person's anatomical configuration and proportions.

Devices for providing massage to selected parts of a person's body are often utilized to relieve stress, pain, tight muscles, or just for enjoyment of the sensation. Further, vibration devices are used for similar purposes. Even more specifically, massage devices having a motorized means for vibrating a massage member may be used both for relaxing sore muscles, but also for stimulating specific anatomical parts of a person's body, such as internal or external genetalia.

Various devices have been proposed in the art having vibration members capable of massaging or vibrating particular parts of the human body, including a person's genital areas. Although assumably effective for their intended purposes, the existing devices are not capable of conforming to the unique anatomical shape and proportion of each person's body. Specifically, the existing devices do not have a handle that is completely adjustable based on anatomy and proportion, also known as an anthropometric handle.

Therefore, it would be desirable to have an anthropometric massage device having a handle that is adjustable so that a vibrating massage member may be manipulated depending on a person's anatomical structure. Further, it would be desirable to have an anthropometric massage device that is waterproof and suitable for massaging both internal and external male or female genitalia.

### SUMMARY OF THE INVENTION

An anthropometric massage device according to the present invention includes a length adjustable handle having first and second portions slidably movable relative to one another for telescopically varying the length of the handle. A shape adjustment member having a plurality of rotatable joints extending between opposed ends thereof for movement between straight and curved configurations. The rotatable joints are secured at selected positions in a friction fit engagement. The massage device includes a stimulation member having opposed ends and a cylindrical configuration defining an open interior space. A vibrating motor is positioned within the interior space of the stimulation member and is electrically connected to a power source, the stimulation member being vibrated when the motor is actuated.

Therefore, a general object of this invention is to provide an anthropometric massage device having a handle and a vibrating massage member.

Another object of this invention is to provide an anthropometric massage device, as aforesaid, in which the handle is adjustable according to a user's anatomical shape and proportion.

Still another object of this invention is to provide an anthropometric massage device, as aforesaid, that is waterproof.

Yet another object of this invention is to provide an anthropometric massage device, as aforesaid, that is battery powered.

A further object of this invention is to provide an anthropometric massage device, as aforesaid, that is length adjustable.

A still further object of this invention is to provide an anthropometric massage device, as aforesaid, that is comfortable and safe to use.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a side view of an anthropometric massage device according to a preferred embodiment of the present invention shown in a generally U-shaped configuration;

FIG. 2 is a front view of the massage device as in FIG. 1 shown in a linear extended configuration;

FIG. 3 is another front view of the massage device as in FIG. 1 shown in an a linear retracted configuration;

FIG. 4 is an exploded view of the massage device as in FIG. 2;

FIG. 5 is a block diagram of the electronic components of the present invention;

FIG. 6a is an exploded view of the handle from a reverse angle as in FIG. 4;

FIG. 6b is an isolated view on an enlarged scale of the handle first portion taken from FIG. 6a;

FIG. 6c is a sectional view taken along line 6c-6c of FIG. 6a; and

FIG. 7 is an isolated view on an enlarged scale taken from a portion of FIG. 6c.

### DESCRIPTION OF THE PREFERRED EMBODIMENT

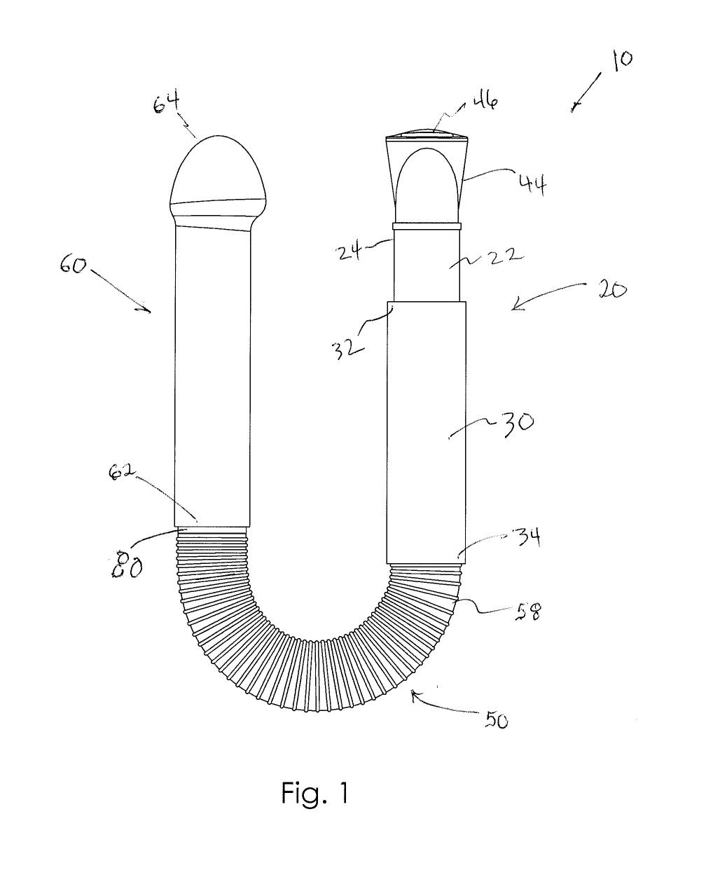

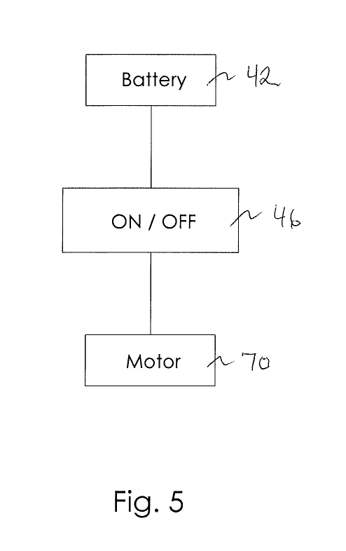

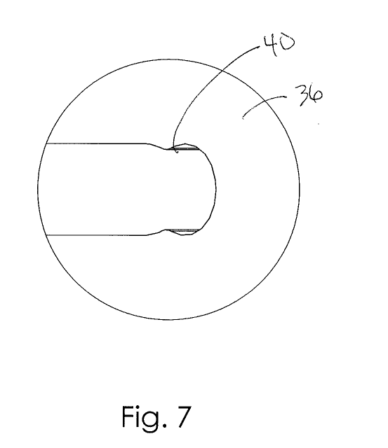

An anthropometric massage device according to a preferred embodiment of the present invention will now be described with reference to FIGS. 1 to 7 of the accompanying drawings. The massage device 10 includes a length adjustable handle 20, shape adjustment member 50, stimulation member 60, and a vibrating motor 70 situated in the stimulation member 60.

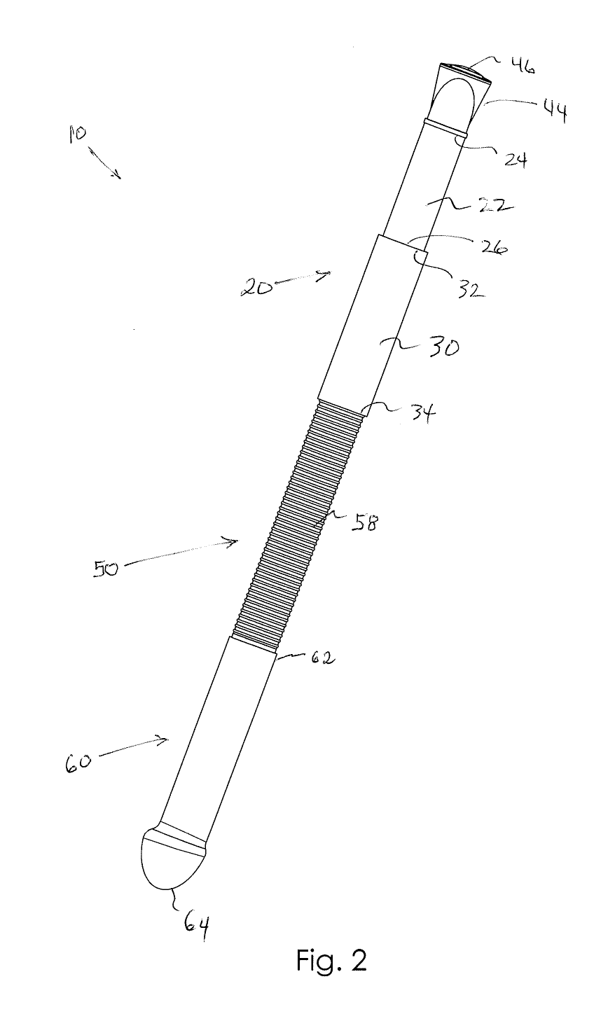

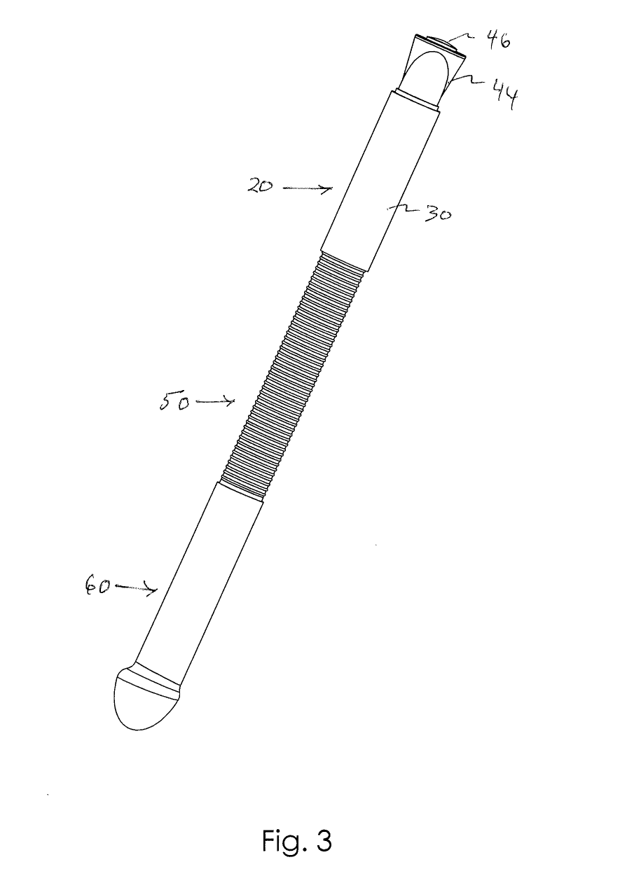

The length adjustable handle 20 includes first 22 and second 30 handle portions that are configured to move slidably relative to one another in a telescopic manner. Preferably, the second portion 30 includes a diameter slightly larger than a diameter of the first portion 22 such that the first portion 22 is selectively receivable into the second portion 30 to provide length adjustment to the handle 20. The length adjustable handle 20 is movable between a retracted configuration (FIG. 3) and a plurality of extended configurations (FIGS. 1 and 2). It is understood that the first 22 and second 30 portions of the handle 20 each include first 24, 32 and second 26, 34 ends, respectively, each end defining an opening through which wires may extend as will be described later.

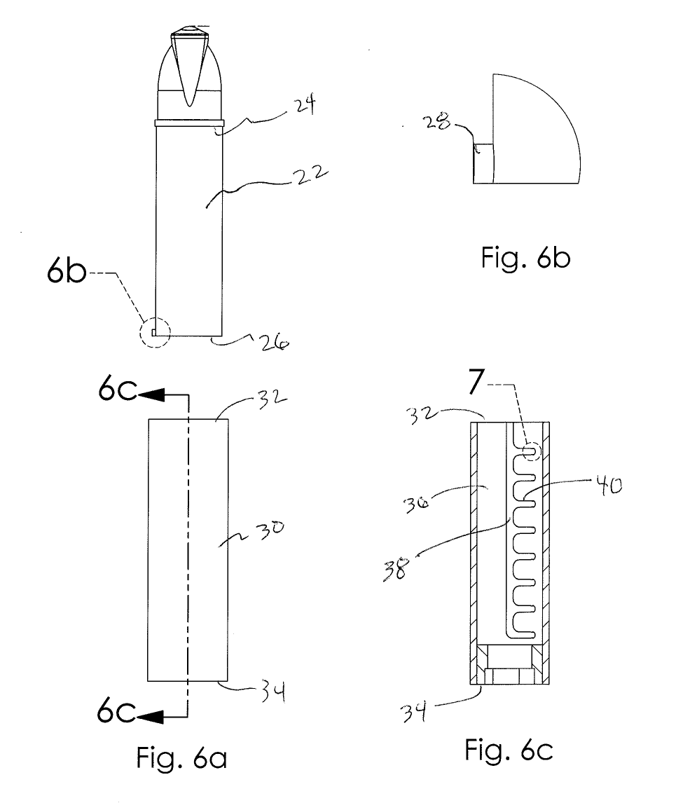

The handle 20 includes means for selectively locking the first 22 and second 30 portions at a desired position relative to one another and, as a result, adjusting an overall length of the device 10 for use. More particularly, the second portion 30 includes an interior wall 36 that defines an elongate channel 38 extending longitudinally from the first end 32 of the second portion 30 substantially to the second end 34 of the second portion 30 (FIG. 6c). The second portion interior wall 36 further defines a plurality of notches 40 each in operative communication with the channel 38. The first portion 22 of the handle 20 includes a nub 28 fixedly mounted to or integrally constructed with an outer wall of the first portion 22, the nub 28 having a configuration that is complementary to that of each notch 40 such that the nub 28 may be received in a respective notch 40 in a gentle friction fit coupling. In other words, the nub 28 may be held securely in a respective notch 40 during use but may be dislodged therefrom upon exertion of a modest pulling or pushing force by a user, such as for changing a length adjustment.

The massage device 10 includes a power source, such as a battery 42, for delivering current as will be further described below. A battery casing 44 may be fixedly attached to the first end 24 of the handle first portion 22. An activation switch 46, such as an on/off button, may be positioned atop the battery casing 44 as shown throughout the accompanying drawings although other positions would also be suitable. Further, it is understood that the activation switch 46 may be merely “on” or “off” or may include variable speed settings.

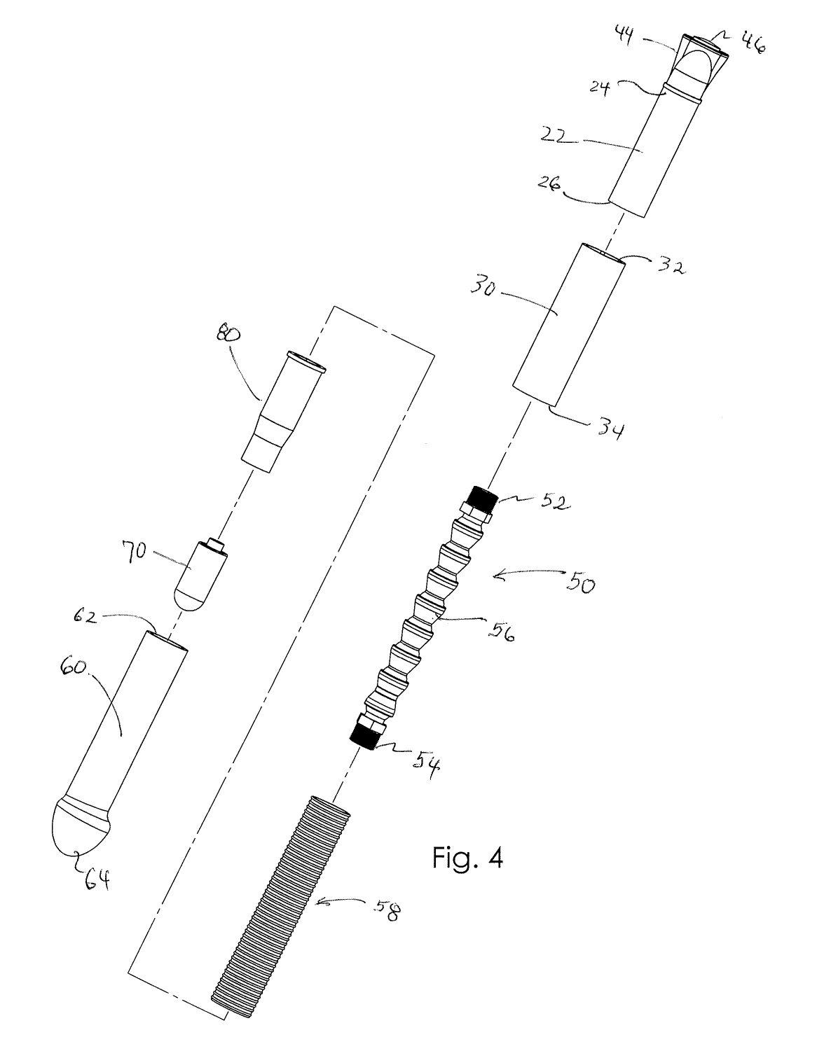

The shape adjustment member 50 includes opposed first 52 and second 54 ends. The first end 52 may be coupled to a second end 34 of the handle second portion 30 such as through a threaded configuration, friction-fit arrangement, or with hardware such as fasteners, clasps, or the like (FIG. 4). The adjustment member 50 includes a plurality of rotatable joints 56, such as ball-and-socket joints, extending between said first 52 and second 54 ends. Each joint includes a generally rounded head (the “ball”) configured to be received in a respective rounded cavity (the “socket”) for rotational movement therein. In fact, rotary movement the joints enables moving the handle 20 between a plurality of configurations. It is understood that each joint may be manufactured at a size that allows a light friction fit to hold the joint at a selected configuration until manually changed. The plurality of rotatable joints 56 enables the massage device 10 to be movable between a linear configuration (FIG. 2), a generally U-shaped configuration (FIG. 1), and numerous configurations therebetween.

A cover 58 may be provided that extends at least between said first 52 and second 54 ends of the shape adjustment member 50 and preferably between the second end 34 of the handle second portion 30 and the stimulation member 60. The cover 58 substantially surrounds the adjustment member 50. Preferably, the cover 58 is constructed of a flexible and waterproof material.

The stimulation member 60 includes proximal 62 and distal 64 ends and presents a generally cylindrical configuration defining an interior space. Outer surfaces of the handle 20, cover 58, and stimulation member 60 include a waterproof material. The distal end 64 of the stimulation member 60 may include a domed shaped configuration indicative of a penis. Further, the outer surface may include a textured surface or even include one or more surface features, such as nubs, ribs, etc., intended to provide greater stimulation when contacted with a person's internal or external genetalia. The stimulation member 60 may include an outer surface that is constructed of silicone, rubber, glass, plastic, or the like.

The vibrating motor 70 may be electrically connected to the battery 42 with wires (not shown). The motor 70 is positioned within the interior space of the stimulation member 60. Preferably, the motor 70 includes a housing having a domed configuration complementary to the domed configuration of the distal end 64 of the stimulation member 60 such that it may be situated therein. When actuated, i.e. receives current from the battery 42, the motor 70 vibrates and, as a result, causes the stimulation member 60 to vibrate. As indicated previously, the battery casing 44 may include a variable speed switch such that the motor 70 may vibrate selectively at variable speeds.

The massage device 10 may include an extension member 80 that is situated between the adjustment member 50 and the motor 70. More particularly, the extension member 80 defines opposed open ends and is sized to be substantially received inside the stimulation member 60. One end of the extension member 80 is fixedly attached to the adjustment member 50 and the opposed end is coupled to the motor 70 inside the stimulation member 60 as is indicated in FIG. 4.

In use, a user may adjust both length and shape of the massage device 10 to conform anthropometrically to the shape and proportion of his or her own body. For instance, the length of the handle 20 may be adjusted by manipulating the first 22 and second 30 portions thereof between completely retracted and multiple extended configurations, as described above. The overall configuration of the massage device 10 may be adjusted to a desired position, such as from a completely linear configuration to a generally U-shaped configuration, and many other intermediate positions. Then, a user may activate the motor 70 by way of the activation switch 46, causing the motor 70 and stimulation member 60 to vibrate. A user may then choose to position the stimulation member so as to massage portions of his or her internal or external genetalia thereby selectively and intentionally generating feelings of either relaxation or tension such as sexual pleasure.

It is understood that while certain forms of this invention have been illustrated and described, it is not limited thereto except insofar as such limitations are included in the following claims and allowable functional equivalents thereof.

What is claimed is:

1. An anthropometric massage device, comprising:
a length adjustable handle having first and second portions that are configured such that said first portion is slidably received in said second portion and movable between retracted and a plurality of extended configurations;
a shape adjustment member having opposed first and second ends, said adjustment member first end being fixedly attached to said handle, said adjustment member having a plurality of ball-and-socket joints extending between said first and second adjustment member ends such that said adjustment member is movable between linear and a plurality of curved configurations;
a stimulation member having proximal and distal ends, said stimulation member having a generally cylindrical configuration defining an interior space;
a power source;
a vibrating motor electrically connected to said power source and positioned inside said interior space of said stimulation member, said motor being configured to vibrate said stimulation member when actuated.

2. The massage device as in claim 1, further comprising a cover extending between said adjustable handle and said stimulation member, said cover being constructed of a flexible material and being configured to surround said shape adjustment member.

3. The massage device as in claim 2, wherein said power source is a battery.

4. The massage device as in claim 3, further comprising:
a battery casing attached to said first portion of said handle and configured to contain said battery; and
a switch positioned on said battery casing and electrically coupled to said battery for selectively actuating current to flow from said battery to said motor.

5. The massage device as in claim 4, wherein said cover member, said first and second portions of said handle, said stimulation member, and said power source housing are waterproof.

6. The massage device as in claim 2, wherein said cover member, said handle, and said stimulation member are constructed of a waterproof material.

7. The massage device as in claim 1, wherein:
said distal end of said stimulation member includes a dome-shaped configuration indicative of a head of a penis; and
said motor includes a housing having a configuration complementary to said stimulation member distal end such that said distal end vibrates when said motor is actuated.

8. The massage device as in claim 4, wherein said switch is a variable speed switch for selectively varying an amount of current actuated to travel from said battery to said motor.

9. The massage device as in claim 4, wherein:
said second portion includes opposed first and second ends defining first and second openings, respectively;
said second portion includes an interior surface defining a guide channel extending longitudinally from said second portion first end substantially to said second portion second end;
said inner wall of said second portion defining a plurality of notches in operative communication with said guide channel;
said first portion include opposed first and second ends;
said first portion defines a nub positioned on an outer surface thereof adjacent said first portion second end; and
said nub being selectively received in a respective notch when said first portion is slidably received in said second portion so as to selectively adjust said adjustable handle between said retracted and plurality of extended configurations.

10. The massage device as in claim 1, wherein:
said second portion includes opposed first and second ends defining first and second openings, respectively;
said second portion includes an interior surface defining a guide channel extending longitudinally from said second portion first end substantially to said second portion second end;
said inner wall of said second portion defining a plurality of notches in operative communication with said guide channel;
said first portion include opposed first and second ends;
said first portion defines a nub positioned on an outer surface thereof adjacent said first portion second end; and
said nub being selectively received in a respective notch when said first portion is slidably received in said second portion so as to selectively adjust said adjustable handle between said retracted and plurality of extended configurations.

11. The massage device as in claim 1, wherein an outer surface of said stimulation member includes a textured surface.

12. The massage device as in claim 10, further comprising an extension member situated between said adjustment member and said motor and being received in said stimulation member, said extension member having a first end fixedly connected to said adjustment member second end of said shape adjustment member and a second end connected to said motor.

13. The massage device as in claim 12, wherein said rotatable joints are ball-and-socket joints.
